(19) United States
(12) Patent Application Publication
(10) Pub. No.: US 2003/0175002 A1
(43) Pub. Date: Sep. 18, 2003

(54) Low loss polymeric optical waveguide materials

(76) Inventors: Mark Andrews, Westmount (CA); Rabah Hammachin, Montreal (CA); Maria Petrucci, Saint-Sauveur (CA); Anthony Roy, Montreal (CA); Veronique Leblanc-Boily, Laval (CA)

Correspondence Address: KNOBBE MARTENS OLSON & BEAR LLP, 2040 MAIN STREET, FOURTEENTH FLOOR, IRVINE, CA, 92614, US

(21) Appl. No.: 10/123,052

(22) Filed: Apr. 12, 2002

Related U.S. Application Data
(60) Provisional application No. 60/342,953, filed on Dec. 20, 2001.

(51) Int. Cl. G02B 6/10; C07D 301/00

(52) U.S. Cl. 385/141; 549/539; 549/541

(57) ABSTRACT

An organic polymeric optical waveguide and methods of making same are described herein. The waveguide can be used in an integrated optical waveguide device. The organic polymeric material can be formed from monomers described herein in ratios to obtain a certain effect.

### RELATED APPLICATION

This application claims the benefit of U.S. Provisional Application No. 60/342,953, filed Dec. 20, 2001.

### FIELD OF THE INVENTION

The present invention relates to optical waveguides and devices that can be prepared from organic polymers.

### BACKGROUND OF THE INVENTION

Dense wavelength division multiplexing is a technique for transmitting data over an optical fiber. Dense wavelength division multiplexing involves multiplexing many different wavelength signals onto a single fiber. Parallel wavelengths can be densely packed and integrated onto a transmission system utilizing multiple, simultaneous, extremely high frequency signals in the 192 to 200 terahertz range. Systems utilizing dense wavelength division multiplexing may suffer from drawbacks, however, including cross-talk (a measure of how well channels are separated) and difficulties in maximizing channel separation (or the ability to distinguish each wavelength).

Dense wavelength division multiplexing systems with sixteen or more channels are commercially available. In order to combine or separate optical signals for dense wavelength division multiplexing, specialized devices are employed. A multiplexer (MUX) takes optical wavelengths from multiple fibers and converges them into a single light beam. A demultiplexer (DEMUX) separates out each of the wavelength components of the light beam and couples them to individual fibers. In a unidirectional system (i.e., a system using a pair of optical fibers) there is a MUX at the sending end of the fiber and a DEMUX at the receiving end of the fiber. In a bidirectional system (i.e., a system using a single optical fiber) there is a multiplexer/demultiplexer (MUX/DEMUX) at each end of the fiber.

A variety of techniques may be employed for multiplexing and demultiplexing. In prism refraction demultiplexing, the light beam impinges on a prism surface, whereby each component wavelength is refracted differently. Each component wavelength exiting the prism is focused by a lens to enter into an end of an optical fiber. By reversing the order of the lens and prism, different wavelengths from multiple fibers may be multiplexed onto a single optical fiber. In multilayer interference filters, a series of thin film filters are cascaded in the optical path. Each filter transmits one wavelength while reflecting others, thus a series of filters may be employed to separate multiple wavelengths. An arrayed waveguide grating (AWG) includes an array of curved-channel waveguides, each waveguide having a different path length. Light enters the input cavity, is diffracted, and enters the waveguide array where the different path lengths introduce phase delays for the different wavelengths in the output cavity where an array of fibers is located. The different wavelengths exhibit maximum interference at different locations corresponding to the output ports of the AWG.

The production of integrated optical elements, such as arrayed waveguide grating (AWGs) and dense wavelength division multiplexer/demultiplexers (DWDMs) is well known. For example, U.S. Pat. No. 6,069,990 to Okawa, et al. discloses one such production method.

There are many materials from which integrated optical elements can be made. In conventional AWGs, such materials include, but are not limited to, pure or doped silica glass, semiconductor compositions, and organic polymers. There are also several methods for using the aforementioned materials to make AWGs. These methods include, but are not limited to, flame hydrolysis deposition (FHD), plasma enhanced chemical vapor deposition (PE-CVD), molecular beam epitaxy (MBE), metallo-organic chemical vapor deposition (MO-CVD), and coating of organic polymers.

Each of these methods has its advantages and disadvantages. FHD has the disadvantage of high temperature processing (on the order of 1000° C. or more). It is also a time consuming process because it requires multiple processing steps, such as repeated stages of thermal annealing and consolidation (densification) of the deposited glass. In addition, FHD utilizes environmentally-sensitive gases, such as chlorosilanes and phosphines, in the glass alloying process, thereby requiring corresponding safety and environmental systems. Moreover, FHD does not lend itself to the coverage of large area substrates, for example, those having a surface area of about 1000 cm² or more. Furthermore, such high temperature processes are not compatible with the manufacture of semiconductor devices such as application-specific integrated circuits (ASICs) because such semiconductor devices are destroyed by the high temperature processes of FHD.

PE-CVD can be practiced at substantially lower temperatures than can FHD, but it has the disadvantage of being a capital-intensive equipment process. PE-CVD requires almost as many steps as does FHD, and usually requires a long period of time for consolidation and densification of the deposited glass in order to be suitable for optical applications.

MBE is a technique for growing crystalline layers of one material (often a semiconductor) deposited on top of another crystalline material. The substrate (supporting) crystal layer imposes its crystal lattice structure closely, if not identically, onto the structure of the deposited material. The technique is employed to fabricate many kinds of semiconductor microelectronic and optoelectronic devices. Major drawbacks of the MBE technique include that it is compatible with a limited range of semiconductor materials, that it is capital-intensive equipment process, and that it often employs environmentally-sensitive gases (e.g., phosphines and arsines).

MO-CVD converts volatile organometallic molecules into semiconductor crystalline materials by moderate to high temperature decomposition of the organometallic species on a heated substrate surface. In general, with an appropriate choice of semiconductor composition, semiconductor materials can be employed to make multiplexers and demultiplexers. For example, silicon can be employed to guide light and to make DWDM devices. The crystalline silicon is typically processed in the manner of the semiconductor devices described above.

Semiconductor materials suffer several disadvantages, however, when employed in integrated optical devices. Because semiconductors are crystalline, the optical properties of light propagating in semiconductor integrated optical devices can depend on the atomic patterning of the crystal. One of the difficulties of semiconductor DWDMs is that the semiconductor materials are birefringent as a result of the dependence on crystal structure. This birefringence is undesirable in optical information technology processing applications. In addition, semiconductor processing for DWDM manufacture is very capital equipment-intensive, making the process expensive when compared with some other methods.

Moreover, semiconductors are not generally preferred because the refractive index of the semiconductor is higher than that of the glass optical fiber with which it must be connected. Such mismatches in the refractive index give rise to Fresnel reflectance losses that degrade the performance of the device by attenuating the transmitted light. To overcome these losses, a semiconductor DWDM is usually connected to an optical amplifier to increase the intensity of the light signal going into and/or out of the DWDM. Like FHD, it is not possible with semiconductor processing to cover very large area surfaces, for example, those on the order of 1000 cm² or larger.

However, the FHD, PE-CVD, integrated optical devices, and semiconductor processes cannot incorporate organic materials directly into the integrated optical devices because the organic material is destroyed by the plasma or by the high temperature processes. Incorporation of organic materials in the integrated optical devices can impart desirable optical properties to the devices. For example, these properties include ease of altering the refractive index of the glass via the organic moiety, optical nonlinearity for fast modulation and switching, and the capability of direct photo-patternability imparted by the photosensitive response of the organic compound.

Organic polymers offer distinct advantages for making integrated optical devices, such as DWDMs. Polymers can be photo-patterned more easily, and photopatterning can be achieved in a variety of polymeric media, including, but not limited to, polyimides, polysiloxanes, polyesters, polyacrylates, and the like.

Moreover, polymers can be coated over large areas and fabricated into patterns using equipment that is less expensive than that required for FHD, PE-CVD, MO-CVD, and MBE. While not as durable as glass, organic polymers can exhibit many of the desirable features of glass, for use in integrated optics devices. Polymers are advantageous for use in integrated optical applications in that the polymers can host organic molecules exhibiting optical nonlinearity for optical modulation and switching of opto-communications and optoelectronic communications signals.

However, some polymers have the disadvantage of being generally less environmentally stable than glasses and semiconductors. Also, many polymers cannot withstand processing temperatures greater than about 100° C. because of the phenomenon of flow above the glass transition temperature (T_{g}) of the polymer. Such flow can cause the optical circuit to change its shape, which can adversely affect the performance of the integrated optical device.

### SUMMARY OF THE INVENTION

A polymeric material for use in preparing DWDMs that exhibits satisfactory thermal and optical stability, glass transition temperature, optical properties, and ease of fabrication is desirable. Organic polymer-based optical waveguides are generally satisfactory in all of these properties, as compared with waveguides prepared by the aforementioned processes of FHD, PE-CVD, MBE and MO-CVD, especially in regards to process capability and cost.

In a first embodiment, a polymeric material is provided, the material including a glycidyl methacrylate monomer and at least one additional monomer such as a 1H,1H-perfluoro-n-octyl acrylate monomer, a 2,2,2-trifluoroethyl methacrylate monomer, and a 1H,1H-perfluoro-n-decyl acrylate monomer.

In one aspect of the first embodiment, the polymeric material further includes a 2,3,4,5,6-pentafluorostyrene monomer.

In one aspect of the first embodiment, the material is formed from 2,3,4,5,6-pentafluorostyrene, 1H,1H-perfluoro-n-octyl acrylate, and glycidyl methacrylate. The weight ratio of 2,3,4,5,6-pentafluorostyrene to 1H,1H-perfluoro-n-octyl acrylate to glycidyl methacrylate may be about 90 to about 10: about 5 to about 40: about 5 to about 25; or about 80 to about 70: about 15 to about 20: about 9 to about 12; or about 7: about 2: about 1.

In one aspect of the first embodiment, the material is formed from 2,2,2-trifluoroethyl methacrylate monomer, 1H,1H-perfluoro-n-decyl acrylate monomer, and glycidyl methacrylate monomer. The ratio of 2,2,2-trifluoroethyl methacrylate to 1H,1H-perfluoro-n-decyl acrylate monomer to glycidyl methacrylate monomer may be about 90 to about 5: about 0.1 to about 1: about 0.5 to about 3; or about 7.5 to about 6.5: about 0.45 to about 0.6: about 1.5 to about 2.2; or about 7.45 to about 0.55 to about 2.

In one aspect of the first embodiment, the material is formed from 2,2,2-trifluoroethyl methacrylate monomer and glycidyl methacrylate monomer. The ratio of 2,2,2-trifluoroethyl methacrylate monomer to glycidyl methacrylate monomer may be about 6 to about 9.5: about 0.5 to about 2.5; or about 8 to about 8.5: about 1.5 to about 1.7; or about 8.35 to about 1.65.

In a second embodiment, an optical device is provided including a polymeric material formed from a glycidyl methacrylate monomer and at least one additional monomer selected from the group consisting of a 2,3,4,5,6-pentafluorostyrene monomer, a 1H,1H-perfluoro-n-octyl acrylate monomer, a 2,2,2-trifluoroethyl methacrylate monomer, and a 1H,1H-perfluoro-n-decyl acrylate monomer. The optical device may be an integrated optical waveguide device. The polymeric material may further include a 2,3,4,5,6-pentafluorostyrene monomer. The optical waveguide device may include a dense wavelength division multiplexing device.

In one aspect of the second embodiment, the device may include a substrate, a buffer layer, a guide layer, and a cladding layer. The guide layer may include a polymeric material formed from 2,3,4,5,6-pentafluorostyrene monomer, 1H,1H-perfluoro-n-octyl acrylate monomer, and glycidyl methacrylate monomer. The buffer layer may include a polymeric material formed from 2,2,2-trifluoroethyl methacrylate monomer, 1H,1H-perfluoro-n-decyl acrylate monomer, and glycidyl methacrylate monomer. The cladding layer may include a polymeric material formed from 2,2,2-trifluoroethyl methacrylate monomer and glycidyl methacrylate monomer.

In a third embodiment, a process for preparing a polymeric material for use in fabricating an optical device is provided, the process including the steps of providing a first monomer including glycidyl methacrylate; providing a second monomer including 2,2,2-trifluoroethyl methacrylate; providing a polymerization catalyst; and polymerizing the monomers, whereby a terpolymeric material suitable for use in fabricating an optical device is obtained.

In one aspect of the third embodiment, the method further includes the step of providing a third monomer including 1H,1H-perfluoro-n-decyl acrylate.

In one aspect of the third embodiment, the polymerization catalyst includes benzoyl peroxide.

In a fourth embodiment, a process is provided for preparing a polymeric material for use in fabricating an optical device, the process including the steps of providing a first monomer including glycidyl methacrylate; providing a second monomer including 2,3,4,5,6-pentafluorostyrene; providing a third monomer including 1H,1H-perfluoro-n-octyl acrylate; providing a polymerization catalyst; and polymerizing the monomers via a free radical polymerization reaction, whereby a polymeric material suitable for use in fabricating an optical device is obtained.

In a fifth embodiment, a polymeric material is provided, the material including a terpolymer of Formula I:

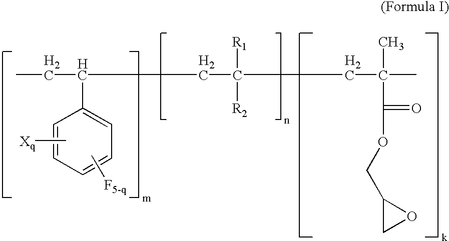

wherein

q is an integer from 0 to 5; p is an integer from 0 to 10; y is an integer from 0 to 4;

R₁ is —CH₃ or H;

R₂ is

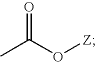

Z is selected from the group consisting of —(CF₂)ₚ—CF₃, —C(CF₃)₂H, and

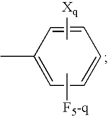

X is selected from the group consisting of H, CF₃,

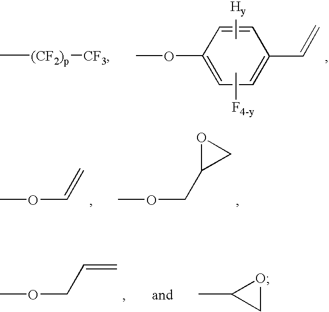

and m, n, and k are non-zero integers.

In aspects of the fifth embodiment, a ratio of m:n:k is about 90 to about 10: about 5 to about 40: about 5 to about 25; or about 90 to about 60: about 10 to about 25: about 5 to about 15; or about 80 to about 70: about 15 to about 20: about 9 to about 12; or about 7: about 2: about 1.

In one aspect of the fifth embodiment, Z is —C(CF₃)₂H.

In one aspect of the fifth embodiment, Z is —(CF₂)ₚ—CF₃ and p is 8.

In one aspect of the fifth embodiment, Z is —(CF₂)ₚ—CF₃ and p is 10.

In one aspect of the fifth embodiment, Z is

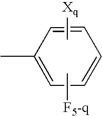

and q is 0.

In one aspect of the fifth embodiment, the terpolymer is a block polymer or a random polymer.

In one aspect of the fifth embodiment, an optical device including the polymeric material of the fifth embodiment is provided. The optical device may be an integrated optical waveguide device.

In a sixth embodiment, a process is provided for preparing a polymeric material for use in fabricating an optical device, the process including the steps of providing a first monomer including glycidyl methacrylate; providing a second monomer of Formula IA:

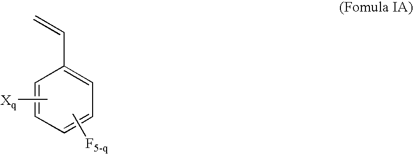

wherein:

X is selected from the group consisting of H, CF₃,

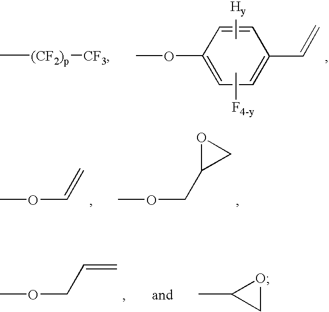

q is an integer from 0 to 5; p is an integer from 0 to 10; and y is an integer from 0 to 4; providing a third monomer of Formula IB:

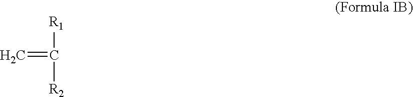

wherein

R₁ is —CH₃ or H;

R₂ is

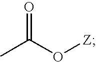

Z is selected from the group consisting of —(CF₂)ₚ13 CF₃, —C(CF₃)₂H, and

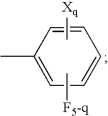

X is selected from the group consisting of H, CF₃,

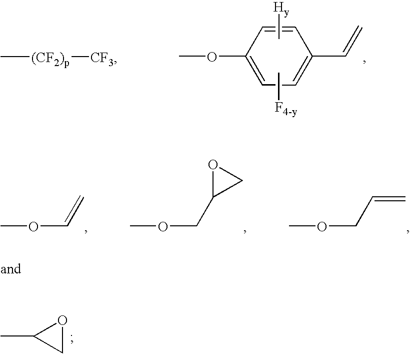

q is an integer from 0 to 5; p is an integer from 0 to 10; and y is an integer from 0 to 4; providing a polymerization catalyst; and polymerizing the monomers via a free radical polymerization reaction, whereby a polymeric material suitable for use in fabricating an optical device is obtained.

In a seventh embodiment, a polymeric material is provided, the material including a terpolymer of Formula II

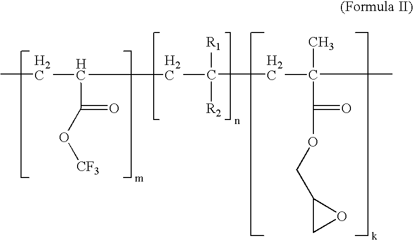

wherein

q is an integer from 0 to 5; p is an integer from 0 to 10; R₁ is —CH₃ or H;

R₂ is

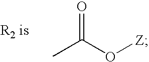

Z is selected from the group consisting of —(CF₂)ₚ—CF₃, —C(CF₃)₂H, and

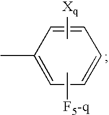

and m, n, and k are non-zero integers.

In aspects of the seventh embodiment, a ratio of m:n:k is about 90 to about 5: about 0.1 to about 1: about 0.5 to about 3; or about 8 to about 6: about 0.3 to about 7: about 1 to about 2.5; or about 7.5 to about 6.5: about 0.45 to about 0.6: about 1.5 to about 2.2; or about 7.45: about 0.55: about 2.

In one aspect of the seventh embodiment, Z is —C(CF₃)₂H.

In one aspect of the seventh embodiment, Z is —(CF₂)ₚ—CF₃ and p is 8.

In one aspect of the seventh embodiment, Z is —(CF₂)ₚ—CF₃ and p is 10.

In one aspect of the seventh embodiment, Z is and q is

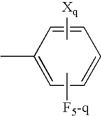

and q is 0.

In one aspect of the seventh embodiment, the terpolymer is a block polymer or a random polymer.

In one aspect of the seventh embodiment, an optical device is provided including the polymeric material of the seventh embodiment. The optical device may include an integrated optical waveguide device.

In an eighth embodiment, a process for preparing a polymeric material for use in fabricating an optical device, the process including the steps of providing a first monomer including glycidyl methacrylate; providing a second monomer of Formula IIA:

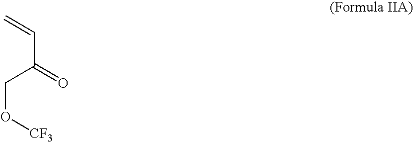

providing a third monomer of Formula IIB:

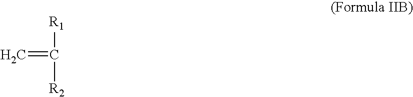

wherein

R₁ is —CH₃ or H;

R₂ is

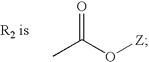

Z is selected from the group consisting of —(CF₂)ₚ—CF₃, —C(CF₃)₂H, and

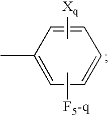

X is selected from the group consisting of H, CF₃,

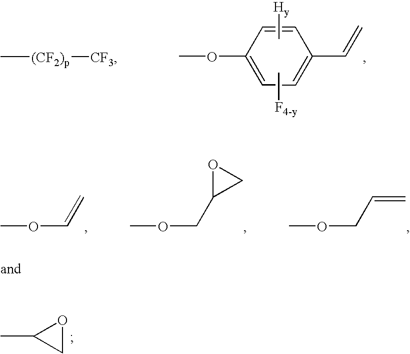

q is an integer from 0 to 5; p is an integer from 0 to 10; and y is an integer from 0 to 4; providing a polymerization catalyst; and polymerizing the monomers via a free radical polymerization reaction, whereby a polymeric material suitable for use in fabricating an optical device is obtained.

In a ninth embodiment, a polymeric material is provided, the material including a copolymer of Formula III:

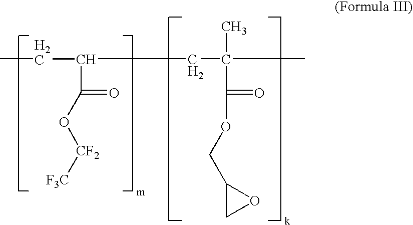

wherein m and k are non-zero integers.

In aspects of the ninth embodiment, the ratio of m:k is about 6 to about 9.5: about 0.5 to about 2.5; or about 7.5 to about 9: about 1 to about 2; or about 8 to about 8.5: about 1.5 to about 1.7; or about 8.35: about 1.65.

In one aspect of the ninth embodiment, the copolymer is a block polymer or a random polymer.

In one aspect of the ninth embodiment, an optical device is provided including the polymeric material of the ninth embodiment. The optical device may be an integrated optical waveguide device.

### BRIEF DESCRIPTION OF THE DRAWINGS

FIG. 1 shows layers of an embodiment of a device comprising a silicon wafer, a buffer layer, a guiding layer, and a cladding layer; and the refractive indices of the layers.

FIG. 2 shows a flow chart of a microfabrication process.

FIG. 3 shows an infrared (IR) spectrum of a glycidyl methacrylate homopolymer, a 2,3,4,5,6-pentafluorostyrene homopolymer, and a terpolymer of 2,3,4,5,6-pentafluorostyrene, glycidyl methacrylate, and 1H,1H-perfluoro-n-octyl acrylate.

FIG. 4 shows IR spectra in the region of the telecommunications windows of a terpolymer of 2,3,4,5,6-pentafluorostyrene, glycidyl methacrylate, and 1H,1H-perfluoro-n-octyl acrylate both before and after UV irradiation.

FIG. 5 shows IR spectra of a terpolymer of glycidyl methacrylate, 2,2,2-trifluoroethyl methacrylate, and 1H,1H-perfluoro-n-decyl acrylate (designated “POLYMER #2)) and a copolymer of glycidyl methacrylate and 2,2,2-trifluoroethyl methacrylate (designated “POLYMER #3”).

### DETAILED DESCRIPTION OF THE PREFERRED EMBODIMENT

The following description and examples illustrate embodiments compatible with the present invention. Those of skill in the art will recognize that there are numerous variations and modifications of the embodiments that are also compatible with the present invention. Accordingly, the description of the embodiments herein should not be deemed to limit the scope of the present invention.

In preferred embodiments, an optical waveguide is provided which incorporates organic polymers. The polymers include, but are not limited to, fluorinated styrenic acrylate components with epoxy functionality. Optical waveguides prepared from such polymers can be employed in integrated optical waveguide devices comprising AWGs, couplers, wavelength division multiplexers (WDMs), such as dense wavelength division multiplexer (DWDM), coarse wavelength division multiplexer (CWDM), and optical devices comprising combinations of these elements.

The performance of an integrated optical waveguide device may be affected by any number of factors, including various linear and nonlinear effects. In Polarization Mode Dispersion (PMD), components of a signal having different polarizations travel at different speeds within an optical fiber or other optical device, resulting in multipath interference when the signal reaches the receiver. PMD becomes more of a problem the longer the distance traveled by the signal, and generally only becomes a problem in long haul systems (i.e., systems wherein the signal travels for more than 500 km).

Other effects include wavelength or chromatic dispersion, wherein optical pulses spread out as they travel over an optical fiber, eventually causing the pulses to overlap, and thereby limiting the rate at which signals may be transmitted over the fiber. Waveguide dispersion occurs because the refractive index difference between the cladding or buffer layer and the guiding layer varies with wavelength. Light of short wavelengths tends to be confined within the guiding layer, such that the effective refractive index is close to the actual refractive index of the guiding material. Light of longer wavelengths spreads into the cladding, however, which results in an effective refractive index close to the actual refractive index of the cladding. Waveguide dispersion may result in propagation delays of certain wavelength components of a signal.

In addition, other nonlinear effects can contribute to signal attenuation. Stimulated Brillouin scattering is caused by back reflection of light due to acoustic waves generated by the interaction of the transmitted light with silica molecules in the optical fiber. Stimulated Brillouin scattering can occur in systems wherein high laser output powers are employed. Rayleigh scattering is the most common form of scattering and results from scattering from variations of the refractive index due to variations in the density of the fiber or optical component. Stimulated Raman scattering results in wavelength shifts of the scattered light as a result of interactions with the silica molecules in a fiber or component. Signal attenuation can result from such scattering, as well as from adsorption processes and stresses on the fiber.

In four-wave mixing, nonlinear interactions among the different channels due to the nonlinear nature of the refractive index of the guiding material create sidebands that result in inter-channel interference. For example, two or more signals of different frequencies can interact, resulting in the formation of an additional frequency, causing cross-talk and a decrease in the signal-to-noise ratio. Impurities or atomic level defects can absorb energy. Absorptive effects generally tend to have a greater effect on longer wavelengths of light (e.g., >1,700 nm) whereas scattering processes tend to have a greater effect on shorter wavelengths (e.g., <800 nm). Fresnel reflections result from discontinuities in the fiber optic system (e.g., splices in a fiber, air gaps, or interfaces between components). Differences in the refractive index at such interfaces result in back reflection of light and associated signal attenuation.

It is therefore preferred to employ materials in fiber optic systems that reduce the signal attenuation due to scattering. Materials of the preferred embodiments can contain fluorine or deuterium atoms. Such materials exhibit low loss in optical power (typically measured in dB), namely, low signal attenuation and low back-reflection of the signal.

Waveguide Structure

Optical waveguides can include a substrate, a buffer layer, a cladding layer, and a guide. As described below, the organic polymers in accordance with embodiments of the present invention may be useful in preparing one or more of these components of the optical waveguide.

Substrate

Optical waveguides are typically fabricated on a substrate comprising a silicon wafer, however, any suitable material may be employed as the substrate. Other suitable substrates include, but are not limited to, silica, glass, polymeric materials, semiconductors, single crystal silicon wafers, borosilicate glasses, polycarbonate, chlorotrifluoroethylene (CTFE), polyetherimide (e.g., the polyetherimide marketed under the tradename ULTEM® 1000 by GE Plastics of Pittsfield, Mass.), MgF₂, CaF₂, crystal quartz, germanium, GaAs, GaP, ZnSe, ZnS, Cu, Al, Al₂O₃, NaCl, KCl, KBr, LiF, BaF₂, thallium bromide (commonly referred to as “KRS-5”), and thallium bromide chloride (commonly referred to as “KRS-6”).

Cladding Layer

The cladding layer is a transparent layer that covers the guiding layer. The cladding layer has a lower refractive index than the guiding layer, so that when light traveling within the guiding layer strikes the boundary between the cladding and the guiding layer at an angle above the critical angle, ø_{C}, the light undergoes total internal reflection, thereby remaining in the guiding layer. The critical angle is defined as follows:

ø_{C}=arcsin (nᵣ/nᵢ)

wherein nᵣ is the refractive index of the cladding layer and nᵢ is the refractive index of the guiding layer. The difference in the refractive index of the cladding layer and the guiding layer need not be large. For example, a difference of 1% in the refractive indices will yield a value for ø_{C} of 82°.

In certain embodiments, the cladding layer can include the organic polymers described below. Other embodiments utilize other materials as are known in the art for the cladding layer. While certain conventional cladding materials that require high temperature processing steps may not be preferred for use, if the issues relating to processing temperature may be overcome, then there is no obstacle to the use of such materials.

Buffer Layer

The buffer layer isolates the optical field in the guiding layer from the substrate. As does the cladding layer, the buffer layer has a lower refractive index than does the guiding layer. In certain embodiments, the buffer layer can include the organic polymers described below. Other embodiments utilize other materials as are known in the art for the buffer layer. Other materials include, but are not limited to, semiconductors, such as gallium arsenides and indium phosphides; ceramic materials, such as ferro-electric materials and lithium niobate; plastics; and composite materials, such as circuit boards and plastic components. While certain conventional buffer layer materials which require high temperature processing steps may not be preferred for use, if the issues relating to processing temperature may be overcome, then there is no obstacle to the use of such materials.

Guide

Light signals travel primarily within the guiding material. The guiding layer has a refractive index greater than that of the buffer layer and cladding layer surrounding it. In certain embodiments, the guiding layer can include the organic polymers described below. Other embodiments utilize other materials as are known in the art for the guiding layer. Other materials include, but are not limited to, semiconductors, silica, silicon, and transparent ceramics. While certain conventional guiding materials that require high temperature processing steps may not be preferred for use, if the issues relating to processing temperature may be overcome, then there is no obstacle to the use of such materials.

Waveguide Device

A typical optical device generally comprises at least three layers: a buffer layer, a guiding layer, and a cladding layer, each of which may comprise a polymer. For each layer, a polymer is synthesized which meets certain criteria, such as, but not limited to, low loss, specific refractive index on slab, chemical stability, thermal stability, photosensitivity, and the like. To make a polymer film that forms a layer in an optical device, the polymer is typically dissolved in a solvent or co-solvents system, then filtered through a 0.2 μm filter and finally spin-coated on a 6 -inch diameter silicon wafer. While this method is generally preferred for most applications due to its simplicity, other polymer deposition methods or substrates, as are known in the art, may be preferred for preparing certain films or layers.

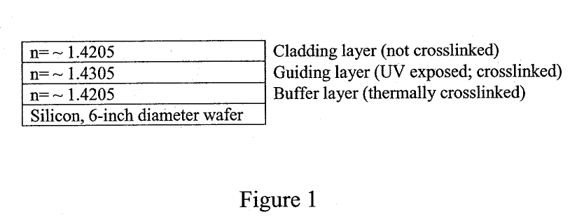

Each layer of the device has a specific Refractive Index (n) so as to achieve Δn=0.01 (Δn=((n_{guiding})−(n_{buffer}))/(n_{cladding})), as shown in FIG. 1.

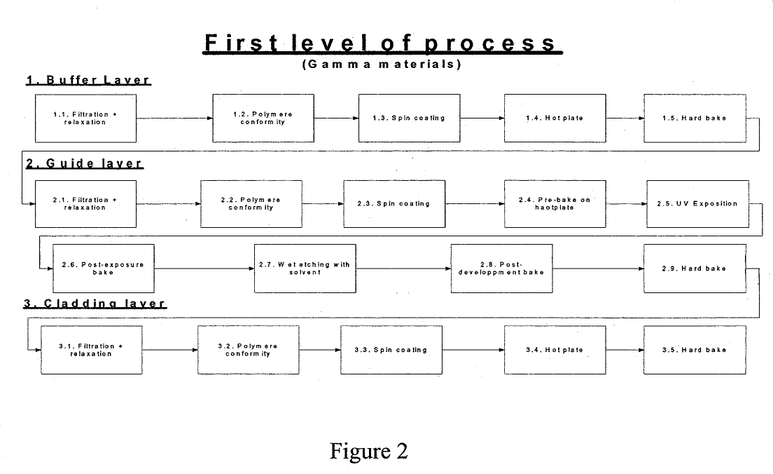

A microfabrication process suitable for use in preferred embodiments of the process is described in FIG. 2. In such a process, a buffer layer solution is prepared, then spin-coated onto a substrate. The substrate is set on a hotplate and the buffer layer is subsequently hard-baked. Next, a guiding layer solution is prepared. The guiding layer solution is spin-coated onto the substrate over the buffer layer. Then, the guiding layer is pre-baked on a hotplate. The guiding layer is subsequently exposed to UV radiation and then goes through a post-exposure bake. Then, the guiding layer is wet-etched. Subsequently, there is a post-development bake and then a hard-bake. Finally, a cladding layer solution is prepared. The cladding layer solution is spin-coated. Then, the material is set on a hotplate and hard-baked.

In one embodiment, the buffer layer is thermally cross-linked in presence of DBU (1,8-diazobicyclo[5.4.0]undec-7-ene). A waveguide is typically cross-linked using a photolithographic technique (for example, UV exposure in presence of a photoinitiator and a photosensitizer) on a guiding layer. Different photomask designs may be employed to create a desired pattern in the layer. A post-exposure-bake is typically conducted to activate polymer densification. Then, a wet-etching with a solvent is performed to remove the portion of the guiding layer that was not cross-linked. Suitable wet-etching solvents may include, but are not limited to, acetate, ketone, alcohol, halogenated organic solvents, such as chloroform or methylene chloride, or an aromatic solvent, such as toluene. The preferred wet etchant may vary depending upon the material to be etched. Other techniques may also be employed to remove the part of the guiding layer (e.g., laser ablation or reactive ion etching). A cladding layer can be tailored to reduce the stress induced polarization dependent loss (PDL) on the waveguides.

Polymeric Materials

As mentioned above, in preferred embodiments, an optical waveguide is provided which incorporates organic polymers. The polymers include, but are not limited to, fluorinated styrenic acrylate components with epoxy functionality. In certain embodiments, base monomers that may be employed to prepare the polymers of preferred embodiments can include moieties such as, for example, fluoro- and/or deutero-substituted carbon atoms, fluoroacrylates, fluoroaryl acrylates, fluorinated alkenyls, chloro- and/or deutero-substituted carbon atoms, chlorinated acrylates, chlorinated aryl acrylates, chlorinated alkenyls, and combinations thereof.

In certain embodiments, it may be preferred that one or more of the base monomers include one or more of the same or different substituents. Preferred substituents include alkyl chains, typically containing from about 2 to about 10 or more carbon atoms. While alkyl groups are generally preferred as substituents, in certain embodiments other hydrocarbyl substituents may also be suitable, including, but not limited to, aryl, alkenyl, cycloalkyl, cycloalkenyl, bicyclic or multicyclic hydrocarbyl groups, branched chains, straight chains, combinations of any of the foregoing, and the like. The hydrocarbyl groups may be substituted or unsubstituted, for example, by one or more heteroatoms. Suitable hydrocarbyl groups may include a range of carbon atoms, for example, 1, 2, 3, 4, 5, 6, 7, 8, 9, 10, or more carbon atoms. Examples of such hydrocarbyl groups include, but are not limited to, propyl, isopropyl, n-butyl, tert-butyl, pentyl, hexyl, heptyl, and higher. In certain embodiments, it may be preferred to substitute one or more hydrogen atoms on the hydrocarbyl group with a deuterium atom.

Deuterium atom or heteroatom-containing substituents may also be present, for example, those containing any halogen (including, but not limited to, fluorine, chlorine, bromine, and iodine), oxygen, and others. The substituents are preferably situated on the acrylate, epoxy, or styrene moieties.

The ratio or ratios of the different monomers in the polymeric materials of preferred embodiments can vary depending upon the properties desired in the polymer. However, any one monomer typically comprises, at a minimum, of from about 5 wt. % or less to about 90 wt. % or more of the polymer, preferably about 6, 7, 8, 9, or 10 wt. % to about 25, 30, 35, 40, 45, 50, 55, 60, 65, 70, 75, 80, or 85 wt. % of the polymer, more preferably from about 11, 12, 13, 14 or 15 wt. % to about 16, 17, 18, 19, 20, 21, 22, 23, 24 or 25 wt. % of the polymer.

The polymers typically possess a T_{g} in the range of about 30° C. or less to 90° C. or higher, preferably from about 35 or 40° C. to about 80 or 85° C., and more preferably from about 40, 41, 42, 43, 44, 45, 46, 47, 48, 49, 50, 51, 52, 53, 54, 55, 56, 57, 58, or 59° C. to about 60, 61, 62, 63, 64, 65, 66, 67, 68, 69, 70, 71, 72, 73, 74, 75, 76, 77, 78, or 79° C.

The preferred molecular weight for the polymer may vary depending upon the processing conditions or device to be fabricated. The molecular weight typically ranges from about 1K or less to about 200K or more, preferably from about 2, 3, 4, 5, 6, 7, 8, 9, or 10K to about 65, 70, 75, 80, 85, 90, 95, 100, 105, 110, 120, 130, 140, 150, 160, 170, 180, or 190K, more preferably from about 11, 12, 13, 14, 15, 16, 17, 18, 19, 20, 21, 22, 23, 24, 25, 26, 27, 28, or 29K to about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60K.

In preferred embodiments, the monomer may not completely react to form a polymer. The amount of unreacted monomer can be up to about 30, 40, 50, or 60 wt. % or more of the total monomer present, preferably less than 25%, and more preferably about 0, 1, 2, 3, 4, 5, 6, 7, 8, 9, 10, or 20 wt. %.

The monomers of the polymer may be arranged in any order, including, but not limited to, arrangements characteristic of block polymers and random polymers.

Polymerization Reaction

Suitable solvents for the polymers of preferred embodiments may include any solvent that is capable of dissolving or dispersing the polymer. The solvent can have a boiling point over about 100, 110, 120, 130, 150, 200, or 250° C. or higher. However, solvents having boiling points of about 100° C. or lower may also be suitable for use in certain embodiments. Examples of preferred solvents include, but are not limited to, acetates, toluene, xylenes, benzenes, and ketones. Preferably, the solvent is a reagent grade solvent.

Catalysts suitable for use in the polymerization reactions of preferred embodiments may include any suitable free radical initiator, for example, peroxides such as benzoyl peroxide and acetyl peroxide, or 2′ azobisisobutyronitrile (AIBN). Ultraviolet radiation or other forms of radiation, such as IR, visible, e-beam, x-ray, and the like, can be employed in the polymerization reaction as well. If ultraviolet radiation is employed in the reaction, the wavelength of the ultraviolet radiation can be about 200 nm or lower to 400 nm or higher; preferably about 200, 210, 220, 230, 240, 250, 260, 270, 280, 290, 300, 310, 320, 330, 340, 390, or 400 nm; more preferably about 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, or 380 nm.

Preferably, polymerization reactions are conducted under an inert atmosphere, such as a nitrogen or argon atmosphere. Preferably, ambient light in the room in which the reaction occurs is UV-filtered. Clean room conditions can be employed for the reactions. Preferably, the clean room is class 100 or class 10000. However, in certain embodiments, it may be preferred to conduct the polymerization reaction under ambient conditions.

The temperature at which the reaction is conducted may depend upon the identity of the initiator employed in the reaction, or other factors. In a preferred embodiment, the initiator is benzoyl peroxide. For embodiments that employ benzoyl peroxide as the initiator, the preferred reaction temperature is about 60° C. or lower to 80° C. or higher, more preferably about 60, 61, 62, 63, 64, 65, 66, 67, 68, 69, 70, 71, 72, 73, 74, 75, 76, 77, 78, 79, or 80° C.

Fabrication Conditions

The fabrication methods using an organic polymer of a preferred embodiment can include, for example, methods of extrusion, deposition, spin coating, spray coating, and dip coating. The patterning of an organic film can include, for example, methods of wet etching, laser ablation, and reactive ion etching.

For microfabrication, any solvent or co-solvent mixture that has a vapor pressure acceptable for the selected method of fabrication can be employed. Preferably, the vapor pressure is less than about 40, 50, or 60 mm Hg at 25° C., more preferably about 0, 5, 10, 15, 20, 25, 30, 35, or 40 mm Hg at 25° C. The boiling point of a solvent suitable for use in microfabrication methods typically varies from about 50° C. or less to 250° C. or more; preferably from about 100 to 180° C.; more preferably from about 130-150° C.; even more preferably about 130, 132, 134, 136, 138, 140, 142, 144, 146, 148, or 150° C.

Microfabrication conditions and polymerization catalysts can include, but are not limited to, photoinitiators, such as iodonium borate salt (available as RHORDOSIL PHOTO INITIATOR® 2074, CAS Reg. No. 178233-72-2, from Rhodia, Inc. of Rock Hill, S.C., referred to herein as “RH 2074”), triarylsulfonium hexafluoroantimonate salts (available as Catalog #407224 from Sigma-Aldrich Canada Ltd., of Oakville, Ontario as a 50% mixture in propylene carbonate, referred to herein as “CD1010”), or [4-[(2-hydroxytetradecyl)oxy]phenyl]phenyliodonium hexafluoroantimonate (CAS Reg. No. 139301-16-9, available as Catalog #445835 from Aldrich Chemical Co., Inc., of Milwaukee, Wis., referred to herein as “CD1012”), combined with a photosensitizer, such as 2-chlorothioxanthen-9-one (CAS Reg. No. 86-39-5, available from Sigma-Aldrich Canada Ltd., referred to herein as “CTX”). The photoinitiator concentration in the solution is typically from about 0.1 wt. % or less to 10 wt. % or more, preferably from about 0.5 to 6%, more preferably from about 3 to 5%, even more preferably about 3, 3.2, 3.4, 3.6, 3.8, 4.0, 4.2, 4.4, 4.6, 4.8, and 5%. The photosensitizer concentration is typically from about 0.1 wt. % or less to 3 wt. % or more, preferably from about 0.1 to 1.2 wt. %, more preferably from about 0.6 to 1.0 wt. %, even more preferably about 0.6, 0.7, 0.8, 0.9, or 1.0%.

DBU is preferably employed for crosslinking of the polymer comprising the buffer layer. The amount of DBU in the polymer solution is typically from about 1 to 15 wt. %, preferably from about 2 to 8 wt. %, more preferably from about 3 to 5 wt. %, even more preferably about 3, 4, or 5 wt. %. In certain embodiments, ethylene diamine (EDA) can be substituted for DBU. In certain embodiments, however, it can be preferable to employ other reagents as are well known to those of skill in the art.

Preferred temperatures for the hard bake of the buffer layer, the pre-bake, post-development bake, or hard bake of the guiding layer, or hard bake of the cladding layer may vary depending upon the polymer forming the layer. However, temperatures in the range of about 400° C. to about 40° C., preferably from about 220° C. to about 50° C., and most preferably from about 190° C. to about 60° C. are preferred for the polymers of preferred embodiments. In certain embodiments, higher or lower temperatures may be preferred.

The time required for completion of the hard-bake of the buffer layer is typically from about 1440 min. to about 30 min., preferably from about 300 min. to about 60 min., and most preferably from about 120 min. to about 60 min.

The time required for the pre-bake of the guiding layer is typically from about 300 sec. to about 10 sec., preferably from about 120 sec. to about 20 sec., and most preferably from about 60 sec. to about 30 sec.

The time required for the post-development of the guiding layer is typically from about 1440 min. to about 30 min., preferably from about 300 min. to about 60 min., and most preferably from about 120 min. to about 60 min.

The time required for the hard bake of the guiding layer is typically from about 1440 min. to about 30 min., preferably from about 300 min. to about 60 min., and most preferably from about 120 min. to about 60 min.

The time required for the hard bake of the cladding layer is typically from about 1440 to about 30 minutes, preferably from about 480 min. to about 30 min., preferably from about 300 min. to about 60 min., and most preferably from about 120 min. to about 60 min.

While the above times for the hard-bake of the buffer layer, the pre-bake of the guiding layer, the post-development of the guiding layer, the hard bake of the guiding layer, the hard bake of the cladding layer, and the hard-bake of the buffer layer are generally preferred, longer or shorter times may be preferred for certain embodiments, depending upon the polymer or other factors.

The wet etch of the guiding layer may be conducted using any suitable etchant, as are known in the art. Particularly preferred etchants include aromatic hydrocarbons, such as toluene and the xylenes, ketones such as acetone, cyclopentanone, esters, and acetates, such as propyl acetate and butyl acetate.

Curing of the guiding layer by exposure to UV radiation is typically conducted according to established curing methods. However, it is generally preferred to employ UV radiation having a wavelength of from about 300 nm to about 450 nm, more preferably from about 300 nm to about 400 nm, and most preferably from about 330 nm to about 370 nm. Any suitable dose may be employed, typically from about 3060 mJ/cm² to about 170 mJ/cm², but more preferably from about 600 mJ/cm² to about 340 mJ/cm². The preferred dose may vary depending upon the wavelength of the UV radiation and the polymer to be cured. It is also generally preferred that the UV radiation have a narrow wavelength distribution, typically from about 300 nm to about 450 nm, preferably from about 350 nm to about 370 nm, and most preferably about 365 nm.

To perform crosslinking, ultraviolet radiation is typically employed. Preferably, the ultraviolet radiation has a wavelength from about 200 or lower to 400 nm or higher; more preferably from about 300 to 380 nm; even more preferably about 300, 305, 310, 315, 320, 325, 330, 335, 340, 345, 350, 355, 360, 365, 370, 375, or 380 nm.

Preferably, microfabrication processes are performed under an inert atmosphere, such as a nitrogen or argon atmosphere. Preferably, the ambient light in the room in which the reaction occurs is UV-filtered. Clean room conditions can be employed for the processes. Preferably, the clean room is class 100 or class 10000. However, in certain embodiments, it may be preferred to conduct the microfabrication process under ambient conditions.

Industry Standard for Device

For a WDM device, specifications of concern include, but are not limited to, insertion loss and uniformity between channels. Typically, insertion loss of a device is preferably about 5 to 7 dB. Polarization dependent loss, which is related to birefringence of a material, is preferably about 0.0004 to 0.0006. Lower molecular weight of the material comprising the optical device may yield a lower birefringence. Typically, the refractive index of the waveguide guiding layer is close to that of the core, and is also lower than the refractive index of the substrate. Adhesion of the waveguide to a substrate or other interface and a resistance to delamination is also preferred.

Glycidyl Methacrylate Materials

In a preferred embodiment, the polymeric materials that form optical devices are selected from a class of glycidyl methacrylate-containing polymeric materials. These materials include guiding terpolymers comprising a (fluoro)styrene moiety, an acrylate moiety, and a glycidyl methacrylate moiety (referred to herein as “TYPE #1 POLYMERS”):

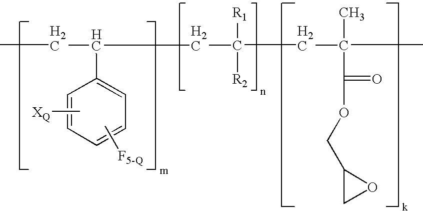

q=0, 1, 2, 3, 4, 5

p=0, 1, 2, 3, 4, 5, 6, 7, 8, 9, 10

R₁═CH₃; H

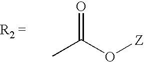

Z=—(CF₂)ₚ—CF₃; —C(CF₃)₂H;

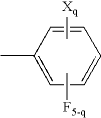

X═H; CF₃; —(CF₂)ₚ—CF₃;

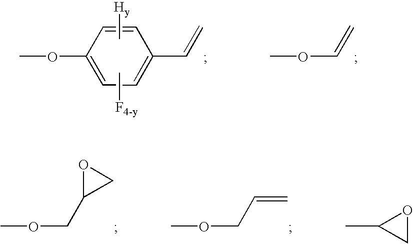

For TYPE #1 POLYMERS depicted above, the ratio (m:n:k) is most preferably (7:2:1). In other embodiments, the ratio of (m:n:k) may typically be (about 10 to about 90: about 5 to about 40: about 5 to about 25), preferably (about 60 to about 90: about 10 to about 25: about 5 to about 15), and more preferably (about 70 to about 80: about 15 to about 20: about 9 to about 12).

A particularly preferred polymer belonging to this class comprises 2,3,4,5,6-pentafluorostyrene (PFS), 1H,1H-perfluoro-n-octyl acrylate (PFOA), and glycidyl methacrylate (GMA) (referred to herein as “POLYMER #1”).

A subset of TYPE #1 POLYMERS that includes other particularly preferred terpolymers comprises a (fluoro)styrene, 1H,1H-perfluoro-i-propyl acrylate (PFIPA), and glycidyl methacrylate (GMA) (referred to herein as “TYPE #1 PFIPA POLYMERS”):

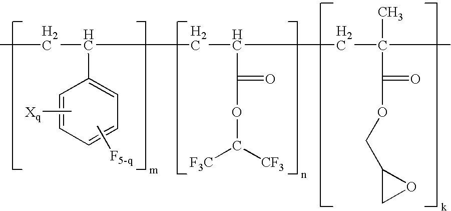

q=0, 1, 2, 3, 4, 5

p=0, 1, 2, 3, 4, 5, 6, 7, 8, 9, 10

y=0, 1, 2, 3, 4

X═H; CF₃; —(CF₂)ₚ—CF₃;

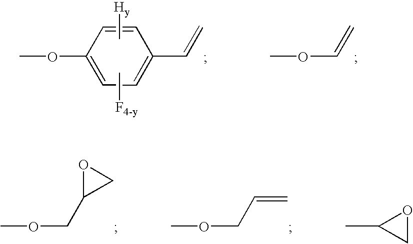

For TYPE #1 PFIPA POLYMERS depicted above, the ratio (m:n:k) is preferably as described for TYPE #1 POLYMERS.

Also included are buffer terpolymers comprising 2,2,2-trifluoroethyl methacrylate (TFEM), an acrylate moiety, and GMA (referred to herein as “TYPE #2 POLYMERS”):

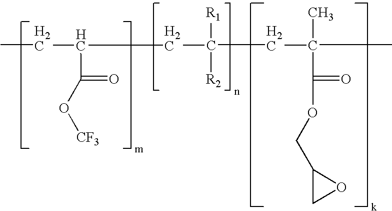

q=0, 1, 2, 3, 4, 5

p=0, 1, 2, 3, 4, 5, 6, 7, 8, 9, 10

y=0, 1, 2, 3, 4

R₁═CH₃; H

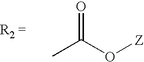

Z=—(CF₂)ₚ—CF₃; —C(CF₃)₂H

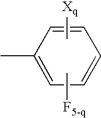

X═H; CF₃; —(CF₂)ₚCF₃;

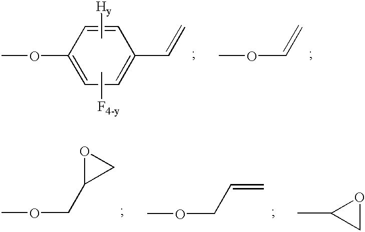

For TYPE #2 POLYMERS depicted above, the ratio (m:n:k) is most preferably (7.45:0.55:2). In other embodiments, the ratio of (m:n:k) may typically be (about 90 to about 5: about 0.1 to about 1: about 0.5 to about 3), preferably (about 8 to about 6: about 0.3 to about 7: about 1 to about 2.5), and more preferably (about 7.5 to about 6.5: about 0.45 to about 0.6: about 1.5 to about 2.2).

A particularly preferred polymer belonging to this class comprises 2,2,2-trifluoroethyl methacrylate (TFEM), 1H,1H-perfluoro-n-decyl acrylate (PFDA), and GMA (referred to herein as “POLYMER #2”):

A cladding copolymer is also provided comprising TFEM and GMA (referred to herein as “POLYMER #3”):

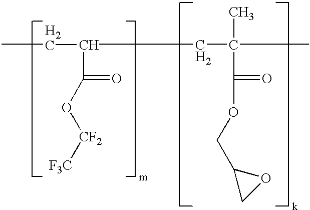

For POLYMER #3 depicted above, the ratio (m:k) is most preferably (8.35:1.65). In other embodiments, the ratio of (m:k) may typically be (about 6 to about 9.5: about 0.5 to about 2.5), preferably (about 7.5 to about 9: about 1 to about 2), and more preferably (about 8 to about 8.5: about 1.5 to about 1.7).

POLYMER #1 Guiding Terpolymer

The POLYMER #1 guiding terpolymer may be prepared according to the following reaction scheme, wherein m is an integer:

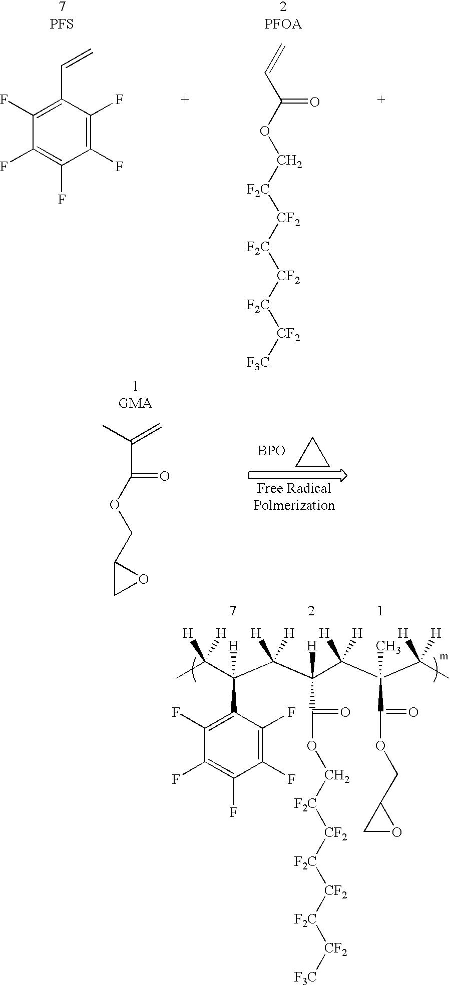

Although benzoyl peroxide (BPO) is employed as an initiator in the scheme shown above and the schemes shown below, other initiators may also be suitable for use.

For the guiding terpolymer as shown in the scheme above, the ratios of the monomers are most preferably about 7 moles PFS to about 2 moles PFOA to about 1 mole GMA. In other embodiments, the ratio of the reagents may typically be from about 90 to about 10 moles PFS to about 5 to about 40 moles PFOA to about 5 to about 25 moles GMA, preferably from about 90 to about 60 moles PFS to about 10 to about 25 moles PFOA to about 5 to about 15 moles GMA, and more preferably from about 70 to about 80 moles PFS to about 15 to about 20 moles PFOA to about 9 to about 12 moles GMA. The monomers polymerize in these ratios to yield a polymer with preferred physical and optical properties desirable in a guiding material. In other embodiments, the ratios of the monomers can be adjusted to obtain polymers with different physical and optical properties.

In the guiding terpolymer, as the proportion of PFS in the polymer increases, the T_{g}, T_{d}, and RI (refractive index) increase while maintaining desirable optical properties in the resulting polymer.

While an increased proportion of GMA provides a greater degree of crosslinking, a large proportion of GMA in the polymer can result in poor optical properties, such as optical loss. Therefore, the proportion of GMA is preferably restricted in order to ensure satisfactory optical properties in the resulting polymer. Also, higher proportions of GMA may result in an increase in the RI.

Increasing the proportion of PFOA may improve optical properties but may also decrease the T_{g}. Therefore, the proportion of PFOA is also preferably restricted in order to ensure satisfactory physical properties.

Other TYPE #1 POLYMER Materials

Other materials that are suitable for use guiding materials or other materials for use in optical devices are shown below:

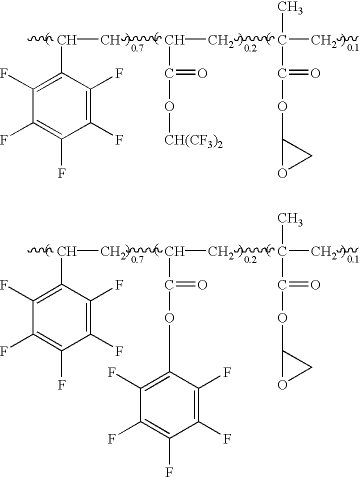

POLYMER #2 Buffer Terpolymer

The POLYMER #2 buffer terpolymer may be prepared according to the following reaction scheme, wherein m is an integer:

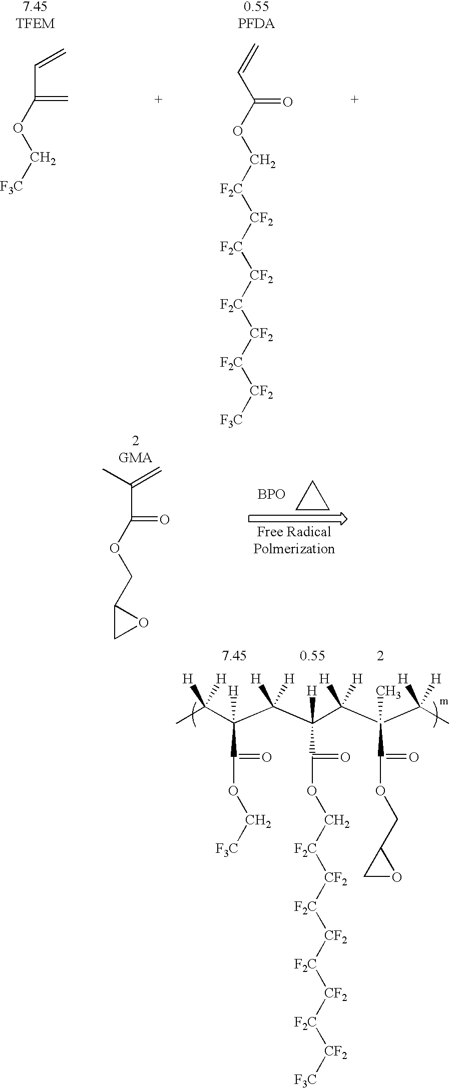

In the buffer terpolymer as shown in the scheme above, the ratio of the monomers are most preferably about 7.45 moles TFEM to about moles 0.55 PFDA to about 2 moles GMA. In other embodiments, the ratio of the monomers may typically be from about 5 to about 90 moles TFEM to about 0.1 to about 1 moles PFDA to about 0.5 to about 3 moles GMA, preferably from about 6 to about 8 moles TFEM to about 0.3 to about 7 moles PFDA to about 1 to about 2.5 moles GMA, and more preferably from about 6.5 to about 7.5 moles TFEM to about 0.45 to about 0.6 moles PFDA to about 1.5 to about 2.2 moles GMA. The monomers polymerize in these ratios to yield a polymer with preferred physical and optical properties desirable in a buffer material. In other embodiments, the ratios of the monomers may be adjusted to obtain polymers with different physical and optical properties. For instance, the ratios can be altered to modify a refractive index of the buffer layer.

In the buffer layer terpolymer, TFEM and PFDA generally function to decrease the RI to achieve Δn=0.01. The GMA improves the cross-linking density and the chemical resistivity of the resulting polymer.

POLYMER #3 Cladding Copolymer

The POLYMER #3 cladding terpolymer may be prepared according to the following reaction scheme, wherein m is an integer:

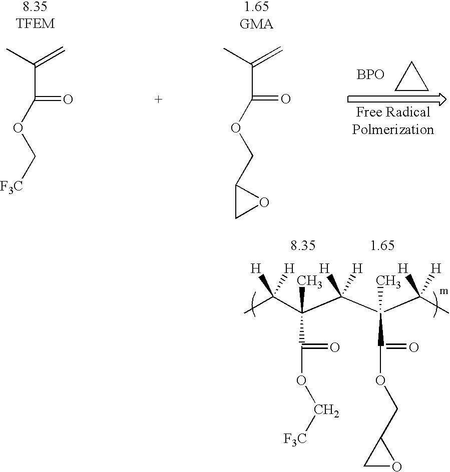

In the cladding copolymer as shown in the scheme above, the ratio of the monomers is most preferably about 8.35 moles TFEM to about 1.65 moles GMA. In other embodiments, the ratio of the monomers may typically be from about 6 to about 9.5 moles TFEM to about 0.5 to about 2.5 moles GMA, preferably from about 7.5 to about 9 moles TFEM to about 1 to about 2 moles GMA, and more preferably from about 8 to about 8.5 moles TFEM to about 1.5 to about 1.7 moles GMA. The monomers polymerize in these ratios to yield a polymer with preferred physical and optical properties desirable in a cladding material. In other embodiments, the ratios of the monomers may be adjusted to obtain polymers with different physical and optical properties. For instance, the ratios can be altered to modify a refractive index of the cladding layer.

In the cladding layer copolymer, the ratios of TFEM and GMA are selected to adjust the RI so as to achieve Δn=0.01.

The disclosure below is of specific examples setting forth methods for making the materials and devices in accordance with preferred embodiments. These examples are not intended to limit the scope, but rather to exemplify preferred embodiments.

### EXAMPLE 1

Synthesis and Preparation of POLYMER #2 Buffer Material Pretreatment of Materials

The materials were purified prior to use in the reactions. Glycidyl methacrylate (GMA) and 1H,1H-perfluoro-n-decyl acrylate (PFDA) were each injected individually into a purification column containing an “inhibitor remover” (Aldrich Cat. No. 30631, HQ/MEHQ). The purity of the reagents was determined by Gas Chromatography-Mass Spectrometry (GC-MS).

Benzoyl peroxide (BPO) was purified by a recrystallization method as follows. Ten grams of BPO was added to 100 ml of methanol. The solution was heated to dissolve the BPO. The solution was cooled to room temperature to allow crystallization of BPO. The recrystallized BPO was collected by vacuum filtration. The BPO was washed with methanol, then air dried for 12 hours. The purity of BPO was determined by High Pressure Liquid Chromatography (HPLC).

Synthesis Procedure

The 2,2,2-trifluoroethyl methacrylate (TFEM, 114.44 grams, 0.68 mole), PFDA (27.85 grams, 0.05 mole), and GMA (25.98 grams, 0.18 mole) were weighed in a flame-dried flask and transferred to a 1 -liter three-necked flask. BPO (4.426 grams, 0.018 mole) was added to the three-necked flask. Then, n-propyl acetate (400 ml) was added to the three-necked flask under a stream of nitrogen. The reaction solution was stirred under a nitrogen atmosphere. After the BPO dissolved, the reaction solution was stirred with flow of nitrogen for 20 minutes before heating. The temperature of the solution was raised to 70°±1° C. over a 1 hour period. The reaction was run for 16 hours at 70°±1° C. The reaction solution was cooled to room temperature. The viscosity of the reaction solution was adjusted to 0-0.5 centipoise (cP) at room temperature by adding n-propyl acetate, as needed.

To precipitate the desired product, the reaction solution was poured into a container filled with methanol. The amount of methanol to precipitate the product was calculated with the aid of the following equation: (volumes of TFEM+PFDA+GMA+n-propyl acetate+n-propyl acetate added for viscosity)×10=volume of methanol. A white polymer precipitated during slow addition of methanol with stirring at room temperature. The polymer was collected by vacuum filtration with a flitted glass filter. The precipitated polymer was washed three times with 300 ml methanol. The polymer was dried for 4 hours using vacuum filtration. Then, the polymer was air-dried for about 12 hours.

The polymer was ground to a fine powder. The powder was dried in a vacuum oven at 50° C. for about 12 hours. The powder was dried until a constant weight within ±0.02 grams was reached. A percent volatility test was performed to check if the weight loss was below 0.2%. If the weight loss was higher than 0.2%, drying was continued until the weight loss was below 0.2%.

The yield of the polymer was approximately 100-135 grams. The resulting polymer was kept in a dessicator. The polymer was stored in a flame-dried hermetic tinted jar.

If there is a problem of precipitating the polymer according to the method described above, the following alternative steps may be performed to precipitate the polymer. The polymer is redissolved in THF at 10 grams of polymer per 200 ml of THF. When the polymer is dissolved, another 200 ml of THF is added. The polymer is precipitated with addition of methanol at 15 times the volume of THF. The polymer is collected by vacuum filtration with fritted glass filter. The polymer is dried for 4 hours using vacuum filtration. Then, the polymer is air-dried for about 12 hours. Finally, the polymer is ground to a fine powder, dried, and tested as described above.

Preparation of POLYMER #2 Buffer Material Solution

The purity of DBU, cyclopentanone, n-propyl acetate and γ-butyrolactone was confirmed by GC-MS.

The POLYMER #2 buffer polymer was dissolved in a mixture of cyclopentanone, n-propyl acetate, and γ-butyrolactone. DBU was added to the solution and the solution stirred. The amounts of the reagents employed for making the solution are calculated, based on the weight of POLYMER #2, as shown in Table 1 below.

TABLE 1

| | | CAT | CAS | |
|---|---|---|---|---|
| Chemicals | Suppliers | number | number | Quantity (g) |
| POLYMER #2 | Lumenon | — | — | W |
| DBU | ALDRICH | 13,900-9 | 6674-22-2 | 4% W |
| Cyclopentanone | ALDRICH | C11,240-2 | 120-92-3 | 0.58* |
| | | | | (W/45%-W) |
| n-Propyl Acetate | SPECTRUM | 13,310-8 | 109-60-4 | 0.27* |
| | | | | (W/45%-W) |
| γ-Butyrolactone | ALDRICH | B10,360-8 | 96-48-0 | 0.15* |
| | | | | (W/45%-W) |

The solution viscosity is preferably approximately 350±50 cP at 21° C., however, in certain embodiments a higher or lower viscosity may be preferred.

The solution was filtered with a 0.20 μm filter. The POLYMER #1 solution was kept in the refrigerator at 0-5° C. in a dry amber jar.

### EXAMPLE 2

Synthesis and Preparation of POLYMER #1 Guiding Material Pretreatment of Materials

The reagents were purified prior to use. 2,3,4,5,6-Pentafluorostyrene (PFS), glycidyl methacrylate (GMA), and 1H,1H-perfluoro-n-octyl acrylate (PFOA) were each injected individually into a purification column containing an “inhibitor remover” (Aldrich Cat. No. 30631, HQ/MEHQ). The purity of the reagents was monitored by GC-MS.

Benzoyl peroxide (BPO) was purified as described above in Example 1.

Synthesis Procedure

The PFS (67.936 grams, 0.35 mole), PFOA (45.412 grams, 0.1 mole), and GMA (7.108 grams, 0.05 mole) were weighed in a flame-dried flask and transferred to a 1 -liter three-necked flask. BPO (6.060 grams, 0.025 mole) was added to the three-necked flask. Then, n-propyl acetate (400 ml) was added to the three-necked flask with a stream of nitrogen. The solution was stirred under a nitrogen flow. After the BPO dissolved, the solution stirred under flow of nitrogen for 20 minutes before heating. The temperature of the reaction solution was raised to 70°±1° C. over a 1 hour period. The reaction was run for 16 hours at 70°±1° C. The reaction solution was cooled to room temperature. The viscosity of the reaction solution was adjusted to 0-0.5 cP at room temperature by adding n-propyl acetate, as needed.

To precipitate the desired product, the reaction solution was poured into a container filled with methanol. The amount of methanol to precipitate the product is calculated with the aid of the following equation: (volumes of PFS+PFOA+GMA+n-propyl acetate+n-propyl acetate added for viscosity)×10 =volume of methanol. A white polymer precipitated from slow addition of methanol at room temperature with stirring. The polymer was collected by vacuum filtration with flitted glass filter. The precipitated polymer was washed three times with 300 ml of methanol. The polymer was dried for 4 hours using vacuum filtration. Then, the polymer was air-dried for about 12 hours.

Finally, the polymer was ground, dried, and analyzed as described above in Example 1.

The yield of the polymer was around 80-100 grams. The resulting polymer was kept in a dessicator. The polymer was stored in a flame-dried hermetic tinted jar.

If there is a problem of precipitating the polymer using the method as described above, the alternative steps described in Example 1 may be employed to obtain the precipitated polymer.

Preparation of POLYMER #1 Guiding Material Solution

CTX and RH 2074 were recrystallized. The purity of CTX and RH 2074 was confirmed by HPLC. The purity of cyclopentanone and n-propyl acetate was verified by GCMS.

The POLYMER #1 guide polymer was dissolved in a mixture of cyclopentanone and n-propyl acetate. RH 2074 and CTX were added to the solution and the solution stirred. The amounts of the reagents employed for making the solution are shown Table 2 below.

TABLE 2

| | | CAT | CAS | |
|---|---|---|---|---|
| Chemicals | Suppliers | number | number | Quantity (g) |
| POLYMER #1 | Lumenon | — | — | W |
| RH 2074 | RHODIA | 911E | NA | 5% W |
| CTX | AVOGADO | 18131-22 | 86-39-5 | 1% W |
| Cyclopentanone | ALDRICH | C11,240-2 | 120-92-3 | 0.7* |
| | | | | (W/47%-W) |
| n-Propyl Acetate | SPECTRUM | P1612 | 109-60-4 | 0.3* |
| | | | | (W/47%-W) |

The solution viscosity is preferably 50±10 cP at 21° C., however, higher or lower viscosities may be preferred for certain embodiments.

The solution was filtered with a 0.20 μm filter. The POLYMER #1 solution was kept in the refrigerator at 0-5° C. in a dry amber jar.

### EXAMPLE 3

Synthesis and Preparation of POLYMER #3 Cladding Material Pretreatment of Materials

The reactants were purified prior to use in the reactions. Glycidyl methacrylate (GMA) was injected into a purification column containing an “inhibitor remover” (Aldrich Cat. No. 30631, HQ/MEHQ). The purity of the GMA was monitored by GC-MS.

Benzoyl peroxide (BPO) was purified as described in Example 1.

Synthesis Procedure

The 2,2,2-trifluoroethyl methacrylate (TFEM, 140.38 grams, 0.83 mole) and GMA (23.46 grams, 0.17 mole) were weighed in a flame-dried flask and transferred to a 1 -liter three-necked flask. BPO (9.688 grams, 0.04 mole) was added to the three-necked flask. Then, cyclopentanone (300 ml) was added to the three-necked flask with a stream of nitrogen. The reaction solution was stirred under a nitrogen flow. After the BPO dissolved, the reaction solution was stirred under a flow of nitrogen for 20 minutes before heating. The temperature of the reaction solution was raised to 60°±1° C. over a 1 hour period. The reaction was run for 4 hours. The reaction solution was cooled to room temperature. The viscosity of the reaction solution was adjusted to 0-0.5 cP at room temperature with cyclopentanone, as needed.

To precipitate the desired product, the reaction mixture was poured into a container filled with methanol. The amount of methanol to precipitate the product is calculated with the aid of the following equation: (volumes of TFEM+GMA+cyclopentanone+cyclopentanone added for viscosity)×10=volume of methanol. A white polymer precipitated during slow addition of methanol with stirring at room temperature. The polymer was collected by vacuum filtration with fritted glass filter. The precipitated polymer was washed three times with 300 ml methanol. The polymer was dried for 4 hours using vacuum filtration. Then, the polymer was air-dried for about 12 hours.

The polymer was ground to a fine powder, dried, and analyzed as described in Example 1.

The yield of the polymer was around 65-95 grams. The resulting polymer was kept in a dessicator. The polymer was preserved in a flame-dried hermetic tinted jar.

If there is a problem of precipitating the polymer using the method as described above, the alternative steps described in Example 1 may be employed to obtain the precipitated polymer.

Preparation of POLYMER #3 Cladding Material Solution

The purity of cyclopentanone was confirmed by GC-MS.

The POLYMER #3 Cladding material polymer was dissolved in cyclopentanone. The amount of cyclopentanone employed for making the solution was calculated based on the weight of POLYMER #3 as shown Table 3 below.

TABLE 3

| | | CAT | CAS | |
|---|---|---|---|---|
| Chemicals | Suppliers | number | number | Quantities (g) |
| POLYMER #3 | Lumenon | — | — | W |
| Cyclopentanone | ALDRICH | C11,240-2 | 120-92-3 | W/37%-W |

The solution viscosity is preferably 500±50 cP at 21° C. However, in certain embodiments a higher or lower viscosity may be preferred.

The solution was filtered with a 0.20 μm filter. The POLYMER #1 solution was kept in the refrigerator at 0-5° C. in a dry amber jar.

### EXAMPLE 4

Characterization of POLYMER #1 Guiding Material

The following Table 4 provides the glass transition temperature (T_{g}) for different samples of POLYMER #1 guiding material. As the molecular weight increased, the value of T_{g} was observed to increase.

TABLE 4

| | T_{g} (° C.) | | | |
|---|---|---|---|---|
| | Not | T_{g} (° C.) | T_{decomposition} | Purity |
| ID | Crosslinked | After UV | (° C.) | (%) |
| POLYMER #1-A | 56 | 73 | 410 | 99.29 |
| (Mw:39K) | | | | |
| POLYMER #1-B | 49 | 66 | 397 | 99.65 |
| (Mw:19K) | | | | |
| POLYMER #1-C | 42 | 65 | — | — |
| (Mw:7K) | | | | |

The following Table 5 shows different molecular weights for samples of POLYMER #1 guiding material. The molecular weight can be fixed in the range of about 7K to 50K or higher.

TABLE 5

| | | |
|---|---|---|
| Mw | Polydispersity | Purity (%) |

| | | | |
|---|---|---|---|
| 50 | K | 1.53 | 95.4 |
| 40 | K | 1.52 | 93.6 |
| 38.3 | K | 1.62 | 95.7 |
| 21.4 | K | 1.73 | — |
| 18.9 | K | 1.66 | — |
| 18.7 | K | 1.86 | 90.8 |
| 7.8 | K | 1.65 | 98.9 |
| 7.3 | K | 1.49 | — |
| 6.6 | K | 1.66 | 99.2 |

The following Table 6 shows mid-IR characteristic peaks for samples of POLYMER #1 guiding material. The peak ratios are compared to show consistency of the synthesis. Improved consistency of the synthesis was observed to correlate with increased molecular weight of the material.

TABLE 6

| | Height | | | | |
|---|---|---|---|---|---|
| | GMA + PFOA | | | | |
| | (C═O) 1759 | PFS (═C—F) | PFAO (—CF₂—) | Ratios | |
| ID | cm⁻¹ | 1655 cm⁻¹ | 659 cm⁻¹ | C═O/═C—F | —CF₂—/═C—F |
| POLYMER #1-A | 0.1006 | 0.0634 | 0.0323 | 1.587 | 0.509 |
| POLYMER #1-B | 0.1667 | 0.0885 | 0.0389 | 1.884 | 0.440 |
| POLYMER #1-C | 0.3482 | 0.1498 | 0.0774 | 2.324 | 0.517 |
| POLYMER #1-D | 0.2886 | 0.1806 | 0.0940 | 1.598 | 0.520 |
| POLYMER #1-E | 0.2782 | 0.1716 | 0.1195 | 1.621 | 0.696 |
| POLYMER #1-F | 0.2493 | 0.1229 | 0.0535 | 2.028 | 0.435 |
| POLYMER #1-G | 0.558 | 0.269 | 0.1092 | 2.074 | 0.406 |
| POLYMER #1-H | 0.2427 | 0.1211 | 0.0589 | 2.004 | 0.486 |

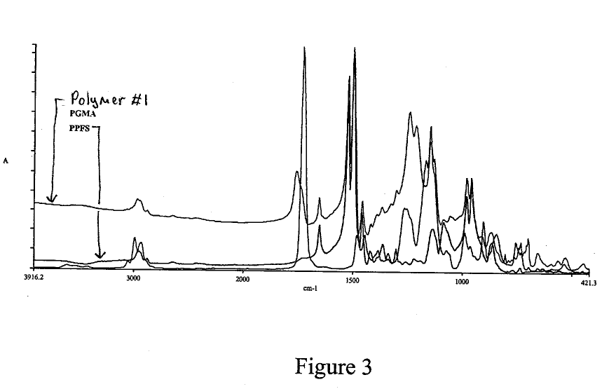

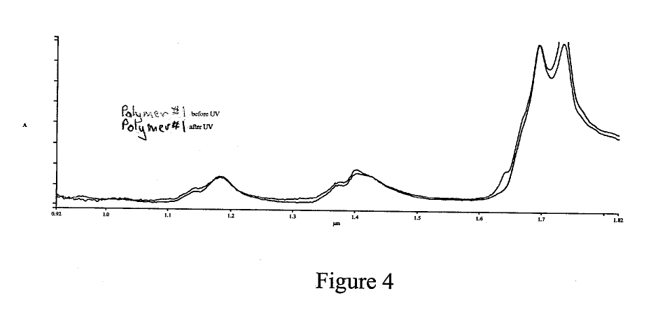

FIG. 3 shows a Fourier Transform InfraRed (FTIR) spectrum of a POLYMER #1 guiding material. A polymer containing GMA as a monomer (poly-GMA, or PGMA) has a characteristic peak at about 1760 cm⁻¹ . A polymer containing PFS as a monomer (poly-PFS, or PPFS) has a characteristic peak at about 1655 cm⁻¹. A terpolymer of POLYMER #1 has the characteristic peaks of PGMA and PPFS and an additional peak at about 660 cm⁻¹ from PFOA. FIG. 4 shows a near-IR spectrum displaying the flatness of a region in a telecommunications window (1.33 μm and 1.55 μm). At 1.64 μm, the epoxy group of the GMA disappears after ultraviolet exposure, which indicates crosslinking of the polymer.

### EXAMPLE 5

Characterization of POLYMER #2 Buffer Layer and POLYMER #3 Cladding Layer

The following Tables 7 and 8 show glass transition temperatures (T_{g}) for different samples of POLYMER #2 buffer layer and POLYMER #3 cladding layer materials.

TABLE 7

| | T_{g} (° C.) | T_{g} (° C.) | T_{decomposition} | Purity |
|---|---|---|---|---|
| ID | (not cross-linked) | (cross-linked) | (° C.) | (%) |
| POLYMER | 60.3 | 80.0 | 262 | 99.98 |
| #2-A | | | | |
| POLYMER | 58.9 | 81.0 | — | — |
| #2-B | | | | |
| POLYMER | — | — | 263 | 99.5 |
| #2-C | | | | |

TABLE 8

| | T_{g} | T_{decomposition} | |
|---|---|---|---|
| ID | (° C.) | (° C.) | Purity (%) |
| POLYMER #3-A | 64 | 255 | 99.67 |

The following Tables 9 and 10 show different molecular weights for samples of POLYMER #2 buffer layer and POLYMER #3 cladding layer materials.

TABLE 9

| ID | Mw | Polydispersity |
|---|---|---|
| POLYMER #2-A | 56.1 K | 1.68 |
| POLYMER #2-B | 62.7 K | 1.73 |
| POLYMER #2-C | 63.8 K | 1.78 |
| POLYMER #2-D | 59.6 K | 1.70 |

TABLE 10

| ID | Mw | Polydispersity |
|---|---|---|
| POLYMER #3-A | 94 K | 1.60 |
| POLYMER #3-B | 106 K | 1.74 |

The following Tables 11 and 12 show mid-IR characteristic peaks for samples of POLYMER #2 buffer layer and POLYMER #3 cladding layer materials.

TABLE 11

| | GMA + FEMA | FEMA + PFDA | |
|---|---|---|---|
| ID | C═O (1750 cm⁻¹) | CF₂ (655 cm⁻¹) | CF₂/C═O |
| POLYMER #2-B | 0.1867 | 0.1038 | 0.556 |
| POLYMER #2-C | 0.1938 | 0.0878 | 0.453 |
| POLYMER #2-D | 0.597 | 0.259 | 0.433 |
| POLYMER #2-E | 0.348 | 0.105 | 0.3015 |

TABLE 12

| ID | C═O | CF₂ | CF₂/C═O |
|---|---|---|---|
| | 1750 cm⁻¹ | 655 cm⁻¹ | |
| POLYMER #3-C | 0.509 | 0.163 | 0.319 |
| POLYMER #3-D | 0.605 | 0.176 | 0.292 |

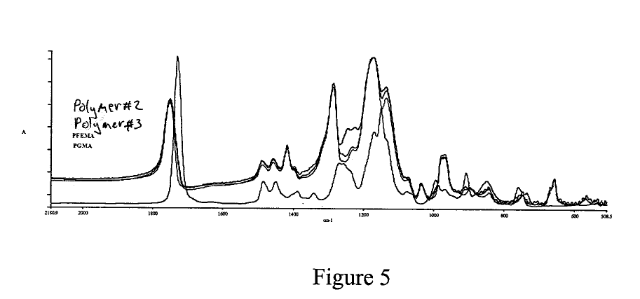

FIG. 5 shows FTIR spectra of POLYMER #2 buffer layer and POLYMER #3 cladding layer materials. FIG. 5 shows that the POLYMER #2 buffer layer comprises TFEM, FFOA, and GMA and that the POLYMER #3 cladding layer comprises TFEM and GMA.

### EXAMPLE 6

Microfabrication Results

Table 13 shows the results for microfabrication of optical devices comprising the polymers of preferred embodiments in terms of refractive index of the slab, thickness, rotation speed and uniformity on the thickness. Similarly, the results of chemistry characterization appear in Table 14 in terms of molar mass, T_{g} and viscosity for the same solution.

TABLE 13

| | | | Final | Uniformity |
|---|---|---|---|---|
| | | Thickness or | rotation | (Std. dev. of |
| | | guide dim. | speed | thickness) |
| Solution | n (slab) | (μm) | (rpm) | (μm) |
| Buffer | | | | |
| (POLYMER #2) | | | | |
| K1-1 | 1.4208 | 13.24 | 1840 | 0.095 |
| K2-4 | 1.4206 | 13.43 | 1200 | 0.034 |
| K2-5 | 1.4207 | 13.88 | 1100 | 0.243 |
| K1-6 | 1.4204 | 13.29 | 1250 | 0.411 |
| K2-7 | 1.4213 | 13.26 | 1250 | 0.113 |
| K2-8 | 1.4208 | 13.66 | 1250 | 0.084 |
| K2-9 | 1.4205 | 13.51 | 1250 | 0.386 |
| Guide | | L / H | | L / H |
| (POLYMER #1) | | | | |
| M1-1 | 1.4292 | 5.81 / 6.50 | 980 | 3.49 / 1.19 |
| M2-2 | 1.4294 | 8.28 / 6.96 | 1300 | 1.95 / 0.108 |
| M2-3 | N/A | 7.22 / 7.10 | 1200 | 0.615 / 0.134 |
| M2-4 | 1.4300 | 7.26 / 6.94 | 1250 | 0.726 / 0.134 |
| M2-6 | 1.4297 | 6.58 / 6.96 | 1350 | 0.099 / 0.142 |
| M2-7 | 1.4302 | 6.82 / 6.89 | 1250 | 0.160 / 0.071 |
| M3-8 | 1.4306 | 6.94 / 7.02 | 1350 | 0.407 / 0.132 |
| M3-9 | 1.4293 | 7.49 / 7.31 | 1300 | 0.622 / 0.045 |
| M3-10 | 1.4296 | 7.21 / 6.94 | 1150 | 0.229 / 0.063 |
| M3-11 | 1.4293 | 6.96 / 7.21 | 1200 | 0.242 / 0.039 |
| Cladding | | | | |
| (POLYMER #3) | | | | |
| N1-1 | 1.4202 | 25.57 / 21.50 | 900 / 2100 | 0.184 / 0.092 |
| N1-3 | 1.4201 | 22.04 | 1200 | 0.206 |
| N1-4 | 1.4203 | 25.65 | 630 | 0.288 |
| N1-7 | 1.4205 | 23.12 | 1000 | 0.277 |

TABLE 14

| | | Viscosity | | | | Poly- | |
|---|---|---|---|---|---|---|---|
| Solution | Polymer | (cP) | nD (sol.) | density | Mw | dispersity | T_{g} |
| Buffer | | | | | | | |
| (POLYMER #2) | | | | | | | |
| K1-1 | PK1-1 | 286.0 | 1.4060 | 1.1077 | 57173 | 1.64 | |
| K2-4 | PK1-1&2 | 377.5 | 1.4263 | 1.1331 | — | — | |
| K2-5 | PK1-2 | 368.2 | 1.4262 | 1.1311 | 56705 | 1.92 | |
| K1-6 | PK1-3 | — | — | — | 58806 | 2.17 | |
| K2-7 | PK1-3 | 492.7 | 1.4262 | 1.1316 | 58806 | 2.17 | |
| K2-8 | PK1-3&4 | 385.0 | 1.4262 | 1.1309 | | | |
| K2-9 | PK1-4 | 366.7 | 1.4264 | 1.1308 | 55068 | 1.77 | |
| Guide | | L / H | | L / H | | | |
| (POLYMER #1) | | | | | | | |
| M1-1 | PM1-1 | 49.7 | 1.4294 | 1.1681 | 19564 | 1.78 | 47.0 |
| M2-2 | PM1-3 | 66.9 | 1.4300 | 1.1803 | 19866 | 2.05 | 49.8 |
| M2-3 | PM1-3 | 63.7 | 1.4301 | 1.1823 | 19866 | 2.05 | 49.8 |
| M2-4 | PM1-4 | 58.2 | 1.4301 | 1.1805 | 19267 | 2.05 | 50.3 |
| M2-6 | PM1-5 | 69.9 | 1.4300 | 1.1803 | 19541 | 2.12 | 45.6 |
| M2-7 | PM1-5 | 65.0 | 1.4301 | 1.1813 | 19541 | 2.12 | 45.6 |
| M3-8 | PM1-7 | 35.0 | 1.4189 | 1.1669 | 18597 | 1.97 | 44.3 |
| M3-9 | PM1-7&8 | 35.7 | 1.4193 | 1.1664 | 19069* | 2.04* | 46.4* |
| M3-10 | PM1-8 | 36.7 | 1.4195 | 1.1660 | 20048 | 2.19 | 48.4 |
| M3-11 | PM1-8&9 | 36.0 | 1.4195 | 1.1665 | 19558* | 2.12* | 48.8* |
| Cladding | | | | | | | |
| (POLYMER #3) | | | | | | | |
| N1-1 | PN1-1 | 470.0 | 1.4360 | 1.0843 | 84075 | 2.31 | |
| N1-3 | PN1-2 | 267.6 | 1.4362 | 1.0830 | 73008 | 2.47 | |
| N1-4 | PN1-2 | 271.0 | 1.4362 | 1.0830 | 73008 | 2.47 | |
| N1-7 | PN1-2,3,4 | 292.4 | 1.4362 | 1.0828 | — | — | |

*indicates a measure that is actually an average of two distinct results

### EXAMPLE 7

Optical Test Measurements

The optical test measurements shown in Table 15 below are for a sample of 10 chips out of 250 chips manufactured (18 wafers with 15 chips each). All manufactured chips were measured, but the 10 represented chips were selected at random to provide a representative sample.

TABLE 15

| | | | | Ave. adj. | Ave. | |
|---|---|---|---|---|---|---|
| | Max IL | Min IL | IL unif. | cross-talk | PDL | Min. BW |
| Chip No. | (dB) | (dB) | (dB) | (dB) | (dB) | @1dB |
| 1 | −6.446 | −5.212 | 1.234 | 20.811 | 2.618 | 0.273 |
| 2 | −6.433 | −4.823 | 1.610 | 17.108 | 2.264 | 0.289 |
| 3 | −6.100 | −5.687 | 1.018 | 20.231 | 2.299 | 0.271 |
| 4 | −5.551 | −4.453 | 1.098 | 21.421 | 2.996 | 0.283 |
| 5 | −7.047 | −6.008 | 1.039 | 16.527 | 2.656 | 0.285 |
| 6 | −5.981 | −5.094 | 0.887 | 23.585 | 2.313 | 0.282 |
| 7 | −7.049 | −4.940 | 2.109 | 22.119 | 2.340 | 0.281 |
| 8 | −6.139 | −4.949 | 1.046 | 18.924 | 2.725 | 0.284 |
| 9 | −6.450 | −4.670 | 1.780 | 22.094 | 2.531 | 0.275 |
| 10 | −5.995 | −4.936 | 1.059 | 22.027 | 2.527 | 0.278 |

The above description provides several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

Every patent and other reference mentioned herein is hereby incorporated by reference in its entirety.

What is claimed is:

1. A polymeric material, the material comprising a glycidyl methacrylate monomer and at least one additional monomer selected from the group consisting of a 1H,1H-perfluoro-n-octyl acrylate monomer, a 2,2,2-trifluoroethyl methacrylate monomer, and a 1H,1H-perfluoro-n-decyl acrylate monomer.

2. The polymeric material of claim 1, further comprising a 2,3,4,5,6-pentafluorostyrene monomer.

3. The polymeric material of claim 2, wherein the material is formed from 2,3,4,5,6-pentafluorostyrene, 1H,1H-perfluoro-n-octyl acrylate, and glycidyl methacrylate.

4. The polymeric material of claim 3, wherein a weight ratio of 2,3,4,5,6-pentafluorostyrene to 1H,1H-perfluoro-n-octyl acrylate to glycidyl methacrylate is about 90 to about 10: about 5 to about 40: about 5 to about 25.

5. The polymeric material of claim 3, wherein a weight ratio of 2,3,4,5,6-pentafluorostyrene to 1H,1H-perfluoro-n-octyl acrylate to glycidyl methacrylate is about 80 to about 70: about 15 to about 20: about 9 to about 12.

6. The polymeric material of claim 3, wherein a weight ratio of 2,3,4,5,6-pentafluorostyrene to 1H,1H-perfluoro-n-octyl acrylate to glycidyl methacrylate is about 7: about 2: about 1.

7. The polymeric material of claim 1, wherein the material is formed from 2,2,2-trifluoroethyl methacrylate monomer, 1H,1H-perfluoro-n-decyl acrylate monomer, and glycidyl methacrylate monomer.

8. The polymeric material of claim 7, wherein a ratio of 2,2,2-trifluoroethyl methacrylate to 1H,1H-perfluoro-n-decyl acrylate monomer to glycidyl methacrylate monomer is about 90 to about 5: about 0.1 to about 1: about 0.5 to about 3.

9. The polymeric material of claim 7, wherein a ratio of 2,2,2-trifluoroethyl methacrylate to 1H,1H-perfluoro-n-decyl acrylate monomer to glycidyl methacrylate monomer is about 7.5 to about 6.5: about 0.45 to about 0.6: about 1.5 to about 2.2.

10. The polymeric material of claim 7, wherein a ratio of 2,2,2-trifluoroethyl methacrylate to 1H,1H-perfluoro-n-decyl acrylate monomer to glycidyl methacrylate monomer is about 7.45 to about 0.55 to about 2.

11. The polymeric material of claim 1, wherein the material is formed from 2,2,2-trifluoroethyl methacrylate monomer and glycidyl methacrylate monomer.

12. The polymeric material of claim 11, wherein a ratio of 2,2,2-trifluoroethyl methacrylate monomer to glycidyl methacrylate monomer is about 6 to about 9.5: about 0.5 to about 2.5.

13. The polymeric material of claim 11, wherein a ratio of 2,2,2-trifluoroethyl methacrylate monomer to glycidyl methacrylate monomer is about 8 to about 8.5: about 1.5 to about 1.7.

14. The polymeric material of claim 11, wherein a ratio of 2,2,2-trifluoroethyl methacrylate monomer to glycidyl methacrylate monomer is about 8.35 to about 1.65.

15. An optical device comprising a polymeric material formed from a glycidyl methacrylate monomer and at least one additional monomer selected from the group consisting of a 2,3,4,5,6-pentafluorostyrene monomer, a 1H,1H-perfluoro-n-octyl acrylate monomer, a 2,2,2-trifluoroethyl methacrylate monomer, and a 1H,1H-perfluoro-n-decyl acrylate monomer.

16. The optical device of claim 15, wherein the optical device is an integrated optical waveguide device.

17. The optical device of claim 15, wherein the polymeric material further comprises a 2,3,4,5,6-pentafluorostyrene monomer.

18. The optical device of claim 15, wherein the optical waveguide device is a dense wavelength division multiplexing device.

19. The optical device of claim 15, wherein the device comprises a substrate, a buffer layer, a guide layer, and a cladding layer.

20. The optical device of claim 19, wherein the guide layer comprises a polymeric material formed from 2,3,4,5,6-pentafluorostyrene monomer, 1H,1H-perfluoro-n-octyl acrylate monomer, and glycidyl methacrylate monomer.

21. The optical device of claim 19, wherein the buffer layer comprises a polymeric material formed from 2,2,2-trifluoroethyl methacrylate monomer, 1H,1H-perfluoro-n-decyl acrylate monomer, and glycidyl methacrylate monomer.

22. The optical device of claim 19, wherein the cladding layer comprises a polymeric material formed from 2,2,2-trifluoroethyl methacrylate monomer and glycidyl methacrylate monomer.

23. A process for preparing a polymeric material for use in fabricating an optical device, the process comprising the steps of:
providing a first monomer comprising glycidyl methacrylate;
providing a second monomer comprising 2,2,2-trifluoroethyl methacrylate;
providing a polymerization catalyst; and
polymerizing the monomers, whereby a terpolymeric material suitable for use in fabricating an optical device is obtained.

24. The process of claim 23, further comprising the step of:
providing a third monomer comprising 1H,1H-perfluoro-n-decyl acrylate.

25. The process of claim 23, wherein the polymerization catalyst comprises benzoyl peroxide.

26. A process for preparing a polymeric material for use in fabricating an optical device, the process comprising the steps of:
providing a first monomer comprising glycidyl methacrylate;
providing a second monomer comprising 2,3,4,5,6-pentafluorostyrene;
providing a third monomer comprising 1H,1H-perfluoro-n-octyl acrylate;
providing a polymerization catalyst; and
polymerizing the monomers via a free radical polymerization reaction, whereby a polymeric material suitable for use in fabricating an optical device is obtained.

27. A polymeric material, the material comprising a terpolymer of Formula I: 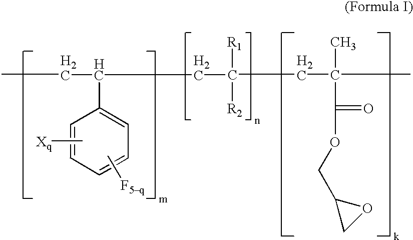
wherein:
q is an integer from 0 to 5;
p is an integer from 0 to 10;
y is an integer from 0 to 4;
R₁ is —CH₃or H;
R₂is 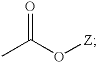
Z is selected from the group consisting of —(CF₂)ₚ—CF₃, —C(CF₃)₂H, and 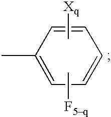
X is selected from the group consisting of H, CF₃, 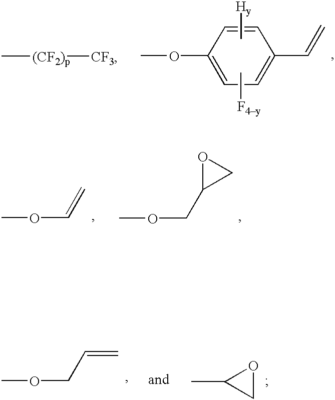
and m, n, and k are non-zero integers.

28. The polymeric material of claim 27, wherein a ratio of m:n:k is about 90 to about 10: about 5 to about 40: about 5 to about 25.

29. The polymeric material of claim 27, wherein a ratio of m:n:k is about 90 to about 60: about 10 to about 25: about 5 to about 15.

30. The polymeric material of claim 27, wherein a ratio of m:n:k is about 80 to about 70: about 15 to about 20: about 9 to about 12.

31. The polymeric material of claim 27, wherein a ratio of m:n:k is about 7: about 2: about 1.

32. The polymeric material of claim 27, wherein Z is —C(CF₃)₂H.

33. The polymeric material of claim 27, wherein Z is —(CF₂)ₚ—CF₃ and p is 8.

34. The polymeric material of claim 27, wherein Z is —(CF₂)ₚ—CF₃ and p is 10.

35. The polymeric material of claim 27, wherein Z is 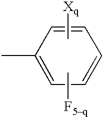
and q is 0.

36. The polymeric material of claim 27, wherein the terpolymer is a block polymer.

37. The polymeric material of claim 27, wherein the terpolymer is a random polymer.

38. An optical device comprising the polymeric material of claim 27.

39. The optical device of claim 38, wherein the optical device is an integrated optical waveguide device.

40. A process for preparing a polymeric material for use in fabricating an optical device, the process comprising the steps of:
a) providing a first monomer comprising glycidyl methacrylate;
b) providing a second monomer of Formula IA 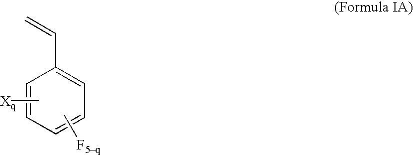
wherein:
X is selected from the group consisting of H, CF₃, 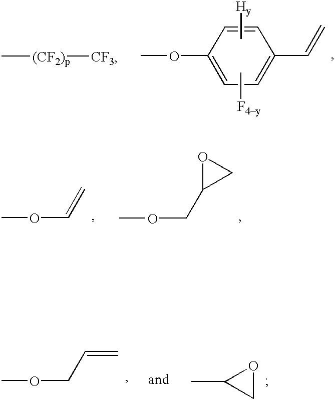
q is an integer from 0 to 5;
p is an integer from 0 to 10; and
y is an integer from 0 to 4;
c) providing a third monomer of Formula IB 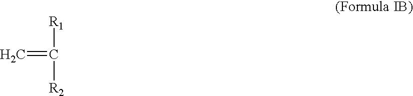
wherein:
R₁ is —CH₃ or H;
R₂ is 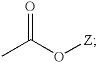
Z is selected from the group consisting of —(CF₂)ₚ—CF₃, —C(CF₃)₂H, and 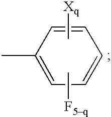
X is selected from the group consisting of H, CF₃, 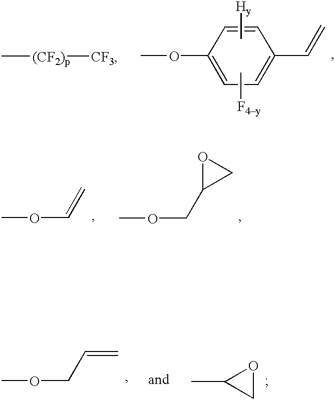
q is an integer from 0 to 5;
p is an integer from 0 to 10; and
y is an integer from 0 to 4;
d) providing a polymerization catalyst; and
e) polymerizing the monomers via a free radical polymerization reaction, whereby a polymeric material suitable for use in fabricating an optical device is obtained.

41. A polymeric material, the material comprising a terpolymer of Formula II 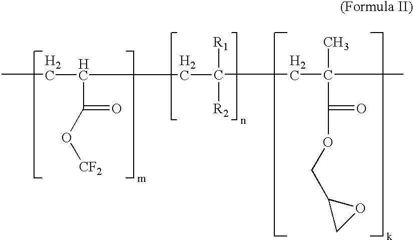
wherein:
q is an integer from 0 to 5;
p is an integer from 0 to 10;
R₁ is —CH₃ or H;
R₂ is 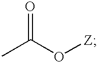
Z is selected from the group consisting of —(CF₂)ₚ—CF₃, —C(CF₃)₂H, and 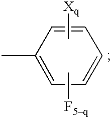
and m, n, and k are non-zero integers.

42. The polymeric material of claim 41, wherein a ratio of m:n:k is about 90 to about 5: about 0.1 to about 1: about 0.5 to about 3.

43. The polymeric material of claim 41, wherein a ratio of m:n:k is about 8 to about 6: about 0.3 to about 7: about 1 to about 2.5.

44. The polymeric material of claim 41, wherein a ratio of m:n:k is about 7.5 to about 6.5: about 0.45 to about 0.6: about 1.5 to about 2.2.

45. The polymeric material of claim 41, wherein a ratio of m:n:k is about 7.45: about 0.55: about 2.

46. The polymeric material of claim 41, wherein Z is —C(CF₃)₂H.

47. The polymeric material of claim 41, wherein Z is —(CF₂)ₚ—CF₃ and p is 8.

48. The polymeric material of claim 41, wherein Z is —(CF₂)ₚ—CF₃ and p is 10.

49. The polymeric material of claim 41, wherein Z is 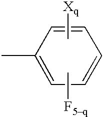
and q is 0.

50. The polymeric material of claim 41, wherein the terpolymer is a block polymer.

51. The polymeric material of claim 41, wherein the terpolymer is a random polymer.

52. An optical device comprising the polymeric material of claim 41.

53. The optical device of claim 52, wherein the optical device is an integrated optical waveguide device.

54. A process for preparing a polymeric material for use in fabricating an optical device, the process comprising the steps of:
a) providing a first monomer comprising glycidyl methacrylate;
b) providing a second monomer of Formula IIA 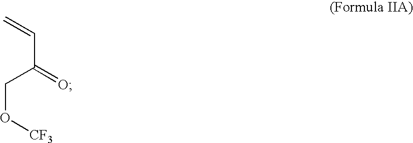
c) providing a third monomer of Formula IIB 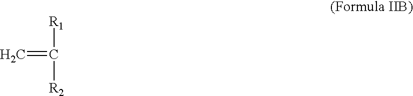
wherein:
R₁ is —CH₃ or H;
R₂ is 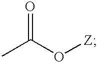
Z is selected from the group consisting of —(CF₂)ₚ—CF₃, —C(CF₃)₂H, and 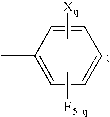
X is selected from the group consisting of H, CF₃, 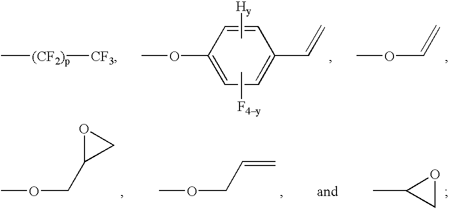
q is an integer from 0 to 5;
p is an integer from 0 to 10; and
y is an integer from 0 to 4;
d) providing a polymerization catalyst; and
e) polymerizing the monomers via a free radical polymerization reaction, whereby a polymeric material suitable for use in fabricating an optical device is obtained.

55. A polymeric material, the material comprising a copolymer of Formula III 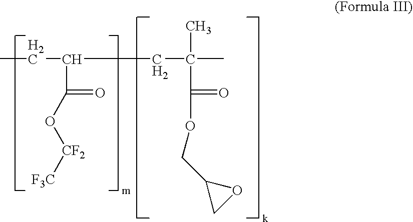
wherein m and k are non-zero integers.

56. The polymeric material of claim 55, wherein a ratio of m:k is about 6 to about 9.5: about 0.5 to about 2.5.

57. The polymeric material of claim 55, wherein the ratio of m:k is about 7.5 to about 9: about 1 to about 2.

58. The polymeric material of claim 55, wherein the ratio of m:k is about 8 to about 8.5: about 1.5 to about 1.7.

59. The polymeric material of claim 55, wherein the ratio of m:k is about 8.35: about 1.65.

60. The polymeric material of claim 55, wherein the copolymer is a block polymer.

61. The polymeric material of claim 55, wherein the copolymer is a random polymer.

62. An optical device comprising the polymeric material of claim 55.

63. The optical device of claim 62, wherein the optical device is an integrated optical waveguide device.
